(19) United States
(12) Patent Application Publication
(10) Pub. No.: US 2025/0235253 A1
(43) Pub. Date: Jul. 24, 2025

(54) MEDICAL DEVICES AND RELATED METHODS FOR DELIVERING ENERGY AND/OR FLUID

(71) Applicants: Boston Scientific Scimed, Inc., Maple Grove, MN (US); Boston Scientific Medical Device Limited, Galway (IE)

(72) Inventors: Katherin NARVAEZ NUÑEZ, Alajuela (CR); Juan Pablo ORTIZ GARCIA, Heredia (CR); Kensuke HAYASHI, Georgetown (MY); Takahiro KOBAYASHI, Tokyo (JP)

(73) Assignees: Boston Scientific Scimed, Inc., Maple Grove, MN (US); Boston Scientific Medical Device Limited, Galway (IE)

(21) Appl. No.: 19/034,157

(22) Filed: Jan. 22, 2025

Related U.S. Application Data
(60) Provisional application No. 63/624,420, filed on Jan. 24, 2024.

(51) Int. Cl. A61B 18/00

(52) U.S. Cl. CPC A61B 18/00; A61B 2018/00077; A61B 2018/00083; A61B 2018/00178; A61B 2018/0091

(57) ABSTRACT

A medical device for delivering energy and/or fluid may comprise: a handle including: a hub for electrically connecting the medical device to an energy source; and a fluid port configured to receive fluid from a fluid source; a shaft including a lumen for transmitting the fluid from the fluid source and a conductor for transmitting energy from the energy source; and an electrode at a distal end of the shaft. The electrode may be electrically coupled to the conductor to deliver energy from the energy source to a tissue. The electrode may include a lumen having a distal opening for delivering fluid from the fluid source to the tissue. The electrode may be immovable relative to the distal end of the shaft.

### CROSS-REFERENCE TO RELATED APPLICATIONS

This application claims the benefit of priority to U.S. Provisional Application No. 63/624,420, filed on Jan. 24, 2024, which is incorporated by reference herein in its entirety.

### TECHNICAL FIELD

Aspects of this disclosure generally relate to medical devices and related methods for delivering energy and/or fluid. Embodiments of the disclosure relate to medical devices and related methods of treating tissue by delivering electrical energy to or into tissue and/or injecting fluid into, under, and/or around tissue with a treatment device that includes an electrode.

### BACKGROUND

Medical devices, such as endoscopes or other suitable insertion devices, are employed for a variety of types of diagnostic and surgical procedures, such as endoscopy, laparoscopy, arthroscopy, gynoscopy, thoracoscopy, cystoscopy, etc. Many of these procedures involve delivering energy to tissue of an organ or a gland to treat lesions (e.g., tumors), infections, and the like. Examples of such procedures include Endoscopic Mucosal Resection (EMR), Endoscopic Sub-mucosal Resection (ESR), Endoscopic Sub-mucosal Dissection (ESD), polypectomy, mucosectomy, etc. In particular, such procedures may be carried out by inserting an insertion device into a subject's body through a surgical incision, or via a natural anatomical orifice (e.g., mouth, vagina, or rectum), and performing the procedure or operation at a target site with an auxiliary device inserted through the insertion device.

### SUMMARY

Examples of the disclosure relate to, among other things, medical devices configured for treating tissue by delivering electrical energy to the tissue.

A medical device for delivering energy and/or fluid may comprise: a handle including: a hub for electrically connecting the medical device to an energy source; and a fluid port configured to receive fluid from a fluid source; a shaft including a lumen for transmitting the fluid from the fluid source and a conductor for transmitting energy from the energy source; and an electrode at a distal end of the shaft. The electrode may be electrically coupled to the conductor to deliver energy from the energy source to a tissue. The electrode may include a lumen having a distal opening for delivering fluid from the fluid source to the tissue. The electrode may be immovable relative to the distal end of the shaft.

Any of the medical devices disclosed herein may have any of the following features, alone or in any combination or subcombination. The sheath may define the lumen. The conductor may be a wire that extends through the lumen. The conductor may include a coil. The coil may extend from the handle to the distal end of the shaft. Windings of the coil may define the lumen of the shaft. The handle may include: proximal channel that is fluidly coupled to the fluid port; and a distal channel. A proximal portion of the coil may be disposed within the distal channel. The distal channel may be in fluid communication with the proximal channel, such that fluid is configured to flow from the fluid port, through the proximal channel, and into the lumen of the shaft. A distal end of the coil may be electrically coupled to the electrode. An outer surface of a portion the coil may include an insulative coating. The insulative coating may terminate proximally of the distal end of the coil. The coil may be electrically coupled to the hub by a band. The band may extend around at least a portion of a circumference of the coil. The medical device may further comprise a distal cap. The distal cap may include an opening extending longitudinally through a portion of the distal cap. A portion of the electrode may extend through the opening of the distal cap. The electrode may be immovable with respect to the distal cap. The electrode may include a widened distal end.

In another example, a medical device for delivering energy and/or fluid may comprise: a shaft including a conductive coil. The coil may include a plurality of windings wound around a central lumen. The medical device may also comprise a distal end including an electrode. The electrode may be electrically coupled to the conductive coil, such that energy transmitted along the conductive coil energizes the electrode.

Any of the devices disclosed herein may include any of the following features, alone or in any combination or subcombination. The electrode may include a lumen having a distal opening. The lumen of the electrode may be in fluid communication with the central lumen of the shaft, such that a fluid flowing through the central lumen of the shaft flows into the lumen of the electrode and exits out of the distal opening of the electrode. A distal end of the coil may contact the electrode.

In another example, a medical device for delivering energy and/or fluid may comprise: a shaft including a conductive coil. The coil may include a plurality of windings wound around a central lumen. The medical device may further comprise a handle including: a hub for electrically connecting the medical device to an energy source; and a fluid port configured to receive fluid from a fluid source. The hub may be electrically coupled to a proximal portion of the conductive coil. The fluid port may be in fluid communication with the central lumen of the shaft.

Any of the medical devices disclosed herein may include any of the following features, alone or in any combination or subcombination. The hub may be electrically coupled to the coil via a conductive band that extends around the coil.

It may be understood that both the foregoing general description and the following detailed description are exemplary and explanatory only and are not restrictive of the disclosure, as claimed.

### BRIEF DESCRIPTION OF THE DRAWINGS

The accompanying drawings, which are incorporated in and constitute a part of this specification, illustrate exemplary aspects of the disclosure and together with the description, serve to explain the principles of the disclosure.

FIGS. 1A and 1B illustrate an exemplary medical device, with FIG. 1A depicting a partially transparent perspective view of a proximal portion of the medical device, and FIG. 1B depicting a partially transparent perspective view of a distal portion of the medical device, according to aspects of this disclosure.

FIGS. 2A and 2B illustrate an alternative exemplary medical device, with FIG. 2A depicting a partially transparent side view of a proximal portion of the medical device, and FIG. 2B depicting a partially transparent side view of a distal portion of the medical device, according to aspects of this disclosure.

### DETAILED DESCRIPTION

Examples of the disclosure include devices and methods for one or more of: facilitating and improving the efficacy, efficiency, and safety of treating and/or manipulating tissue when, for example, applying electrical energy to tissue with an electrode; delivering fluid into, under, and/or around tissue during a medical procedure through the distal end of the electrode; and cutting, resecting, or otherwise treating tissue. Aspects of the disclosure may provide the user with the ability to apply electrical energy or heat to tissue using a medical device having an electrode, and to deliver fluid into and/or under tissue with the same medical device. Aspects of the disclosure may provide the user with the ability to apply electrical energy or heat, and also deliver fluid without having to switch or swap out end effectors. Aspects of the disclosure may help the user penetrate a layer of tissue (e.g., a submucosal layer) to cause perforation or otherwise cut, cauterize, or treat tissue. Aspects of the disclosure may help the user cut, resect, or otherwise remove tissue or other material. Some aspects of the disclosure may be used in performing an endoscopic, laparoscopic, arthroscopic, gynoscopic, thoracoscopic, cystoscopic, or other type of procedure.

For example, the disclosed devices may include a handle, a shaft, and an electrode at a distal end of the shaft. The distal electrode may include a lumen to deliver a fluid into, under, and/or around tissue. The electrode may be electrified in order to cut, cauterize, or otherwise treat tissue. The electrode may be fixed with respect to the shaft. In other words, in all configurations, a distal end of the electrode may extend a same distance from a distal end of the shaft. The electrode may be stationary and not movable (e.g., immovable proximally and distally) with respect to the shaft.

Reference will now be made in detail to examples of the disclosure described above and illustrated in the accompanying drawings. Wherever possible, the same reference numbers will be used throughout the drawings to refer to the same or like parts.

The terms “proximal” and “distal” are used herein to refer to the relative positions of the components of an exemplary medical device. When used herein, “proximal” refers to a position relatively closer to the exterior of the body of a subject or closer to a user, such as a medical professional, holding or otherwise using the medical device. In contrast, “distal” refers to a position relatively further away from the medical professional or other user holding or otherwise using the medical device, or closer to the interior of the subject's body. Proximal and distal directions are labeled with arrows marked “P” and “D”, respectively, throughout various figures. As used herein, the terms “comprises,” “comprising,” “having,” “including,” or other variations thereof, are intended to cover a non-exclusive inclusion, such that a device or method that comprises a list of elements does not include only those elements, but may include other elements not expressly listed or inherent thereto. Unless stated otherwise, the term “exemplary” is used in the sense of “example” rather than “ideal.” As used herein, the terms “about,” “substantially,” and “approximately,” indicate a range of values within +/−10% of a stated value.

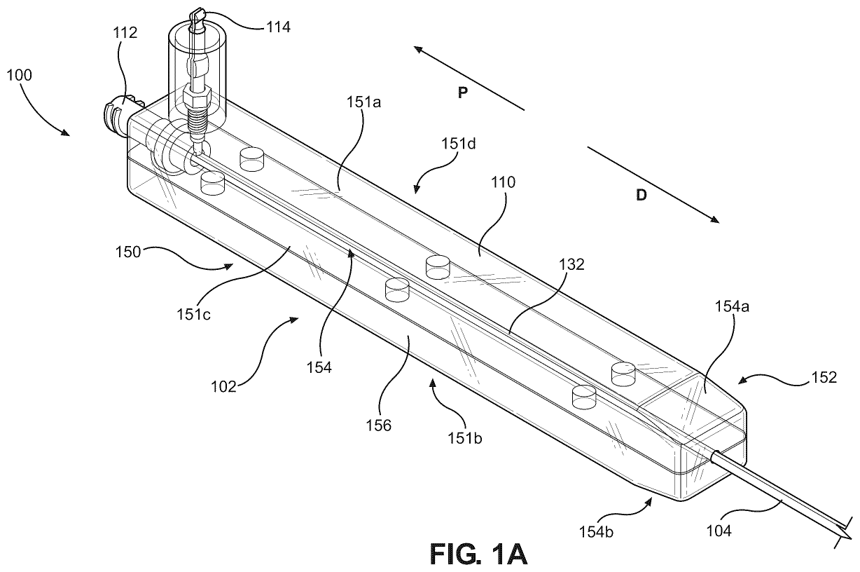

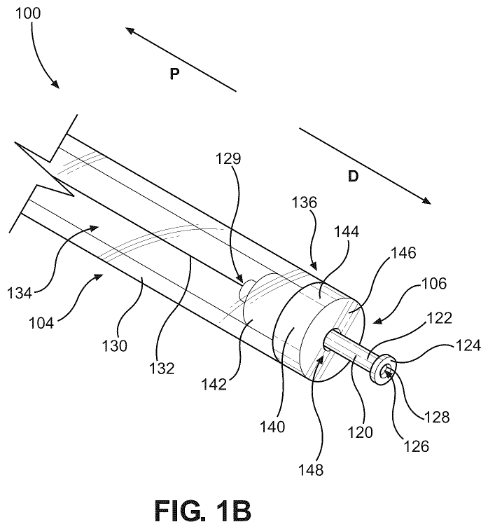

FIGS. 1A and 1B depict a medical device 100 that includes a handle 102 (FIG. 1A), a shaft 104, and a distal end 106 (FIG. 1B). Handle 102 may include a body 110. Handle 102 also may include or otherwise be coupled to or adjacent to a port 112 configured to receive fluid. Handle 102 may also include or otherwise be coupled to a hub 114 configured to receive electrical energy, similar to an electrical plug or socket. Distal end 106 may include an electrode 120. Electrode 120 may (i) be conductive to convey and deliver energy from hub 114 and/or (ii) convey fluid from port 112 to tissue or other material at a treatment site. Electrode 120 may be fixed (i.e., immobile or stationary) relative to shaft 104, as described in further detail below.

As shown particularly in FIG. 1B, shaft 104 may include a sheath 130 and a conductor, such as a wire 132. Sheath 130 may be formed of a flexible, insulating material (e.g., plastic), such that shaft 104 may be inserted into a delivery device (e.g., an endoscopic scope, a sheath, etc.) and advanced to a target site. Sheath 130 may include any suitable number of layers, having any suitable properties. In some examples, shaft 104 may include a plurality of sheaths (e.g., inner sheaths and outer sheaths), although only one sheath 130 is shown in FIG. 1B.

A lumen 134 may extend longitudinally through shaft 104. For example, lumen 134 may extend along an entire length of shaft 104, from handle 102 to distal end 106. As shown in FIG. 1B, in particular, lumen 134 may be defined by an inner surface of sheath 130. Alternatively, where shaft 104 includes a plurality of sheaths, a surface of another sheath may define lumen 134. Lumen 134 may have a uniform width/diameter along a length of shaft 104, or a width/diameter of lumen 134 may vary along the length of shaft 104.

Wire 132 may extend through lumen 134. As described in further detail below, wire 132 may be electrically/conductively coupled to hub 114, such that energy received by hub 114 may travel along wire 132. Although a single wire 132 is depicted, it will be appreciated that device 100 may include a plurality of wires 132. Wire 132 may extend along an entire length of shaft 104 and into handle 102, as described below. Additionally, although not shown, one or more portions of wire 132 may be coated with an insulating material. In some aspects, the insulating coating on wire 132 may help to isolate the electrically charged wire from the fluid being delivered to the treatment site through lumen 134, as discussed in further detail below.

With continued reference to FIG. 1B, distal end 106 may include electrode 120 and a distal cap 140. Cap 140 may be electrically insulating. In some aspects, cap 140 may be formed of rubber or another biocompatible and at least partially flexible material. Cap 140 may include a proximal portion 142 and a distal portion 144. Proximal portion 142 may be positioned within a distalmost end of sheath 130. An outer width/diameter of proximal portion 142 may be approximately the same as an inner width/diameter of sheath 130. Proximal portion 142 may be fixedly coupled to sheath 130 (e.g., using adhesive or another mechanism).

Distal portion 144 of cap 140 may have a larger width/diameter than proximal portion 142 of cap 140. For example, distal portion 144 may have an outer width/diameter that may be approximately the same as an outer width/diameter of sheath 130. Distal portion 144 of cap 140 may be distal to a distalmost end of sheath 130. For example, a proximal face of distal portion 144 may abut a distalmost end of sheath 130. A distal face 146 of distal portion 144 may form a distalmost face of device 100 (except for portions of electrode 120 that extend distally of distal portion 144, as described below).

Cap 140 may be fixed in all directions with respect to shaft 104. In other words, cap 140 may be axially/longitudinally, rotationally, and laterally immovable with respect to shaft 104 and, in particular, a distalmost end 136 of shaft 104. Any suitable portion (e.g., proximal portion 142 or distal portion 144) of cap 140 may be fixedly coupled via adhesive or another mechanism to distalmost end 136 of shaft 104.

Electrode 120 may extend through a channel 148 of cap 140. Electrode 120 may include a shaft 122 and a distal tip 124. Distal tip 124 may extend radially outward from a distal end of shaft 122. Distal tip 124 may have a greater diameter/width than shaft 122. In other words, distal tip 124 may be a widened distal end of electrode 120. A lumen 126 may extend through electrode 120, to a distal opening 128 on a distal face of distal tip 124. Lumen 126 of electrode 120 may be in fluid communication with lumen 134 of shaft 104. For example, as shown in FIG. 1B, a proximal end 129 of shaft 122 may extend proximally of a proximalmost end of proximal portion 142 of cap 140, into lumen 134 of shaft 104. Lumen 126 of electrode 120 may have a proximal opening on a proximal end of proximal end 129 of shaft 122. Alternatively, shaft 122 may include one or more side openings (not shown). Thus, fluid may flow from lumen 134 of shaft 104 into lumen 126 of electrode 120. The fluid may then exit out of distal opening 128 of electrode 120. Alternatively, a proximalmost end of shaft 122 may be within cap 140, and cap 140 may define a channel/lumen (e.g., channel 148) that is in fluid communication with lumen 134 of shaft 104 and lumen 126 of electrode 120, such that lumen 126 of electrode 120 is in fluid communication with lumen 134 of shaft 104 via cap 140.

It is noted that, in other embodiments, instead of fluid being delivered through electrode 120, the fluid may be delivered around electrode 120 (e.g., through openings in cap 140 and/or through channel 148). In some aspects, however, electrode 120 (e.g., electrode shaft 122) may help to guide the fluid to the treatment site, for example, via capillary action.

Wire 132 may be electrically coupled/connected to electrode 120 (e.g., to shaft 122). For example, wire 132 may be coupled to proximal end 129 of shaft 122 using any suitable mechanism (e.g., solder, conductive adhesive, etc.). Electrode 120 may be conductive, such that electrical energy traveling through wire 132 may be delivered to electrode 120, thereby energizing electrode 120, including shaft 122 and/or distal tip 124. Additionally, although not shown, one or more portions of electrode 120 may include insulation, for example, radially surrounding one or more portions of electrode 120.

Electrode 120 may be fixedly coupled to cap 140. For example, electrode 120 may be fixedly coupled to channel 148 or another portion of cap 140 via adhesive or another connection mechanism. In other words, electrode 120 may be immovable or stationary relative to cap 140 and shaft 104. Distal tip 124 may have a fixed axial/longitudinal distance with respect to distal face 146 and distalmost end 136 of shaft 104. In other words, electrode 120 may have a fixed length that extends distally from cap 140/shaft 104.

With reference to FIG. 1A, body 110 may have an elongated shape along a longitudinal axis of body 110. A proximal portion 150 of body 110 may have an approximately rectangular or square cross-sectional shape, such that proximal portion 150 may be approximately a rectangular prism. For example, proximal portion 150 may have a first wall 151a, a second wall 151b, a third wall 151c, and a fourth wall 151d. At a distal portion 152 of body 110, opposing top and bottom walls 154a, 154b of distal portion 152 body 110 (walls on opposite sides of the central longitudinal axis of body 110) may taper radially inward in a distal direction, toward a central longitudinal axis of body 110. It will be appreciated that handle 102 may be rotated, and that the terms “top” and “bottom” are used for ease of reference and refer to the view of FIG. 1A. Distal portion 152 may also have two side walls 156 (only one of which is visible in FIG. 1A) that may be planar and may extend along an entire length of body 110, along proximal portion 150 and distal portion 152. Handle 102 may lack any actuator for any movement of any element of shaft 104 or distal end 106 relative to other elements of shaft 104 or distal end 106.

Hub 114 may be positioned on one side of proximal portion 150. For example, hub 114 may be positioned on a top side of proximal portion (a same side of body 110 that has tapered top wall 154a). Wire 132 may receive energy from hub 114. For example, hub 114 may include one or more pins or prongs to electrically connect/couple to the energy source. The energy source may be an electrocautery source, a radio frequency generator, a heating source, a current generator, etc. In some examples, hub 114 may be removably coupleable to a cord (e.g., via pins or prongs), which may transmit energy from the energy source. Hub 114 may be electrically coupled to wire 132 via any suitable connection (e.g., soldering, conductive adhesive, etc.). In some examples, wire 132 may extend from electrode 120, through shaft 104, and into handle 102, to hub 114. Alternatively, intermediate wires, cables, or other conductive elements may extend between hub 114 and wire 132.

In one aspect, medical device 100 may be used for monopolar electrosurgery, and may include a return electrode positioned remotely from electrode 120 on or otherwise adjacent to the subject. In another aspect, medical device 100 may be used for bipolar electrosurgery. In that instance, electrode 120 may include an active electrode portion, and a return electrode may be provided at or near another portion of electrode 120 and/or shaft 104. In one example, although not shown, two conductive elements (e.g., wires 132) may run through shaft 104, where the conductive elements may be electrically isolated from each other, allowing one to conduct energy to the active electrode and the other to conduct energy from a return electrode. Although not shown, in another aspect, the energy source may be a part of handle 102 (e.g., an internal battery in handle 102).

Handle 102 may include fluid port 112. As shown in FIG. 1A, fluid port 112 may be on a proximal end of handle 102. Fluid port 112 may be couplable to a fluid source such as, for example, a syringe, a pump (not shown), a reservoir, etc., containing a fluid. Fluid port 112 may be in fluid communication with lumen 134 of shaft 104, such that fluid from fluid port 112 may flow into lumen 134. Accordingly, shaft 104 may deliver fluid to the treatment site.

In some examples, shaft 104 may extend proximally through handle 102, such that a proximal end of shaft 104 is coupled to fluid port 112. Alternatively, shaft 104 may have a proximalmost end at a distal end of handle 102, or shaft 104 may extend proximally only partially into handle 102. In such examples, handle 102 may include a channel 154 for receiving wire 132 and fluid from fluid port 112. Alternatively, channel 154 may receive shaft 104, including sheath 130. In a further alternative, channel 154 may be omitted from handle 102.

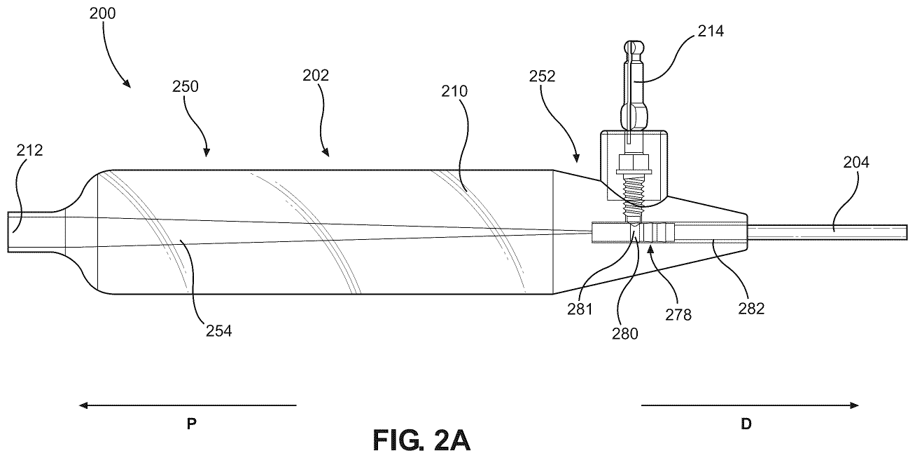

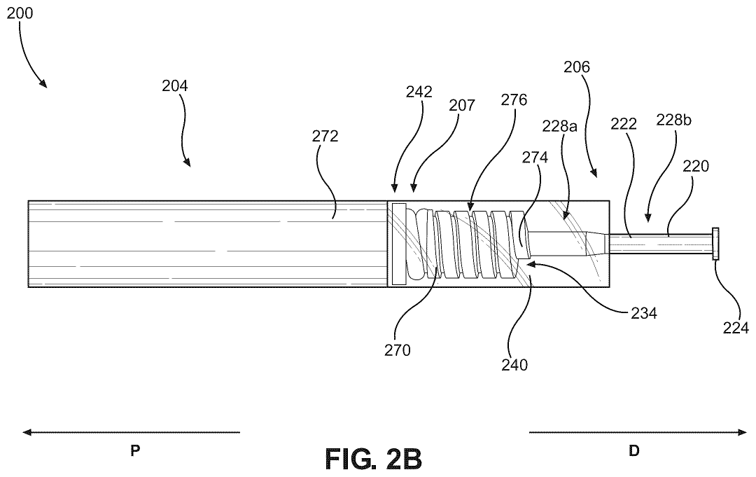

FIGS. 2A and 2B depict an alternative medical device 200. Unless otherwise specified herein, medical device 200 may have any of the features of medical device 100. Like medical device 100, medical device 200 may include a distal electrode 220 for delivering energy and/or fluid to a treatment site. Instead of using a wire (like wire 132) and a sheath 130, medical device 200 may have a conductor, such as a conductive coil 270, which may deliver energy to an electrode 220 to energize electrode 220.

Medical device 200 may have a handle 202 (FIG. 2A), a shaft 204, and a distal end 206 (FIG. 2B). Distal end 206 may include electrode 220. Handle 202 may a body 210. Handle 202 also may include a port 212 configured to receive fluid. Handle 202 may also include or otherwise be coupled to a hub 214 configured to receive electrical energy, similar to an electrical plug or socket. Electrode 220 may be conductive to convey and deliver energy from hub 214 and/or fluid from port 212 to tissue or other material at a treatment site. Electrode 220 may be fixed (i.e., immobile or stationary) relative to shaft 204, as described in further detail below.

With reference to FIG. 2B, distal end 206 may include electrode 220. Electrode 220 may include any of the features of electrode 120, discussed above. Similarly, electrode 120 may include any of the features of electrode 220. Similarly to electrode 120, electrode 220 may include a shaft 222 and a distal end 224. Although not depicted in FIG. 2B, electrode 220 may include a lumen and opening similar to lumen 164 and opening 128, described above. Shaft 222 may have a proximal portion 228a and a distal portion 228b. Distal portion 228b may have a narrower diameter/width than proximal portion 228a.

Shaft 204 may include a conductive coil 270, which may extend from handle 202 to distal end 206. Coil 270 may include a metallic material, such as one or more of stainless steel, Nitinol (nickel titanium alloy), titanium cobalt chrome, silver, platinum or platinum alloys. An outer surface (e.g., a radially outer surface) of coil 270 (and/or other surfaces of coil 270) may be covered with an insulative coating 272. Additionally or alternatively, coil 270 may be covered with an insulative sheath, having any of the properties of sheath 130. Coil 270 and coating 272 may be dimensioned so that shaft 204 has an outer diameter of approximately 2.0 mm to approximately 2.8 mm, or approximately 2.4 mm. Coil 270 may be flexible to allow shaft 204 to bend and curve as device 200 is navigated to a treatment site (e.g., using an insertion device, such as an endoscopic scope).

Coil 270 and coating 272 may replace sheath 130 and wire 132 of device 100. Coil 270 may both transmit energy from hub 214 (as described in further detail below) and may provide structure to shaft 204. In alternatives, coil 270 may extend only partially along a length of shaft 204 (e.g., along only a proximal or distal portion of shaft 204). Other portions of shaft 204 may include features similar to shaft 104 (e.g. include a wire and/or other components, etc.), described above.

As shown in FIG. 2B, a distal end 274 of coil 270 may be electrically coupled to proximal portion 228a of shaft 222 of electrode 220. Distal end 274 of coil 270 may contact electrode 220 (e.g., proximal portion 228a). For example, distal end 274 of coil 270 may be soldered, welded, or coupled via conductive adhesive to shaft 222 of electrode 220. In examples, shaft 222 may be welded directly to an inside surface of coil 270 (a surface that defines a lumen 234, discussed below). In other examples, a small wire may be welded to an inside surface of coil 270 and another end of the small wire may be welded to shaft 222. Thus, energy transmitted through coil 270 from hub 214 may be transmitted to and delivered by electrode 220. Distal end 274 may be positioned such that electrode 220 may extend approximately along a central longitudinal axis of shaft 204 (i.e., a central longitudinal axis of electrode 220 may extend approximately coaxial to a central longitudinal axis of shaft 204).

Windings of coil 270 may define central lumen 234 extending through a center of coil 270, approximately parallel to a central longitudinal axis of coil 270. In other words, a wire of coil 270 may wind around lumen 234 (windings of coil 270 may be wound around lumen 234). Fluid from port 212 may flow into lumen 234, as described below. In some examples, an inner surface of coil 270 may be coated with an insulative material so as to help to prevent fluid in lumen 234 from being exposed to energy carried through coil 270. Alternatively, such insulative material may be omitted, and fluid in lumen 234 may be exposed to energy in coil 270. In a further alternative, a tube may extend through lumen 234 of coil 270 to carry fluid from port 212.

A distal cap 240 may be disposed over a distal portion of coil 270 and proximal portion 228a of shaft 222 of electrode 220. Cap 240 may include an insulating material (e.g., rubber, plastic, polymer, etc.) Coating 272 may be absent from a distal portion 276 of coil 270 that is covered by cap 240. In other words, coating 272 may terminate proximally of distal portion 276 of coil 270. As shown in FIG. 2B, a portion of coil 270 covered by proximal end 242 of cap 240 may have coating 272, such that coating 272 and cap 240 overlap for a portion (e.g., a small portion) of cap 240. Alternatively, coating 272 may terminate distally, at, or proximally of proximal end 242 of cap 240. In some examples, cap 240 may help to keep fluid from leaking out of distal end 206, except at designated locations (e.g., a distal opening of electrode 220). Cap 240 may also reinforce a coupling of coil 270 to electrode 220 (either a direct coupling or a coupling using a small wire, as described above).

As with electrode 120, electrode 220 may have a fixed, unchanging length with respect to a distal portion of shaft 204. In other words, electrode 220 may not be axially (or laterally or rotationally) movable with respect to shaft 204. A distance from distal end 224 to a distalmost end 207 of shaft 204 may be the same in all configurations of device 200. Electrode 220 may be stationary, immovable, or fixed with respect to distalmost end 207 end of shaft 204.

Unless otherwise specified herein, handle 202 may have any of the features of handle 102. Similarly, handle 102 may have any of the features of handle 202, unless otherwise specified. Handle 202 may have a proximal portion 250 and a distal portion 252. Body 210 at proximal portion 250 of handle 202 may have an approximately cylindrical outer shape. At distal portion 252 of handle 202, body 210 may have an approximately cone or frustum outer shape.

Similarly to handle 102, fluid port 212 may be disposed on a proximalmost end of handle 202. For example, fluid port 212 may extend proximally from a proximalmost end of proximal portion 250. Fluid port 212 may have any of the properties of fluid port 112. A central longitudinal axis of fluid port 212 may be approximately coaxial with a central longitudinal axis of handle 202. As shown in FIG. 2A, fluid port 212 may have a smaller width/diameter than adjacent portions of handle 202; however, such a shape and size is merely exemplary.

As shown in FIG. 2A, fluid port 212 may be fluidly coupled to a proximal channel 254 extending through proximal portion 250 of handle 202 and into distal portion 252 of handle 202. As shown in FIG. 2A, proximal channel 254 may have a tapered width/diameter, such that a proximal end of proximal channel 254 has a width/diameter that is approximately the same as a width/diameter of fluid port 212 and a distal end of proximal channel 254 has a width/diameter that is approximately the same as (or smaller than) a width/diameter of lumen 234 of shaft 204. In other words, proximal channel 254 may taper radially inward, toward a longitudinal axis of handle 202, from a proximal end of proximal channel 254 to a distal end of proximal channel 254. Proximal channel 254 may be configured to transmit fluid provided through fluid port 212 to lumen 234, in order to deliver fluid using distal end 206 of device 200.

Whereas hub 114 of handle 102 may be disposed on proximal portion 150 of handle 102, hub 214 of handle 202 may be disposed on distal portion 252 of handle 202. Hub 214 may extend approximately perpendicularly relative to the central longitudinal axis of handle 102. A proximal portion 278 of coil 270 may be electrically coupled/connected to hub 214 via, for example, one or more bands 280 (e.g., crimped conductive bands). Bands 280 may include barbs, and may be formed of conductive material. Bands 280 may be coupled to a proximal portion 278 of coil 270, which may lack coating 272. In other words, proximal portion 278 of conductive coil 270 may be exposed. Hub 214 (e.g., an active cord) or another element (e.g., a cord of an energy source) may be screwed into the handle until it touches and presses on band 280 (e.g., one or more barbs 281 of crimped band 280).

Proximal portion 278 of coil 270, as well as adjacent portions of shaft 204, may be received/positioned/disposed within a distal channel 282 of handle 202. Distal channel 282 may have a uniform width/diameter and may be sized and shaped so as to accommodate coil 270/shaft 204. For example, distal channel 282 may have a slightly larger diameter than an outer diameter of coil 270/shaft 204.

Band(s) 280 may extend around at least a portion of a circumference or perimeter of coil 270. For example, band(s) 280 may be ring-shaped. Band(s) 280 may locally increase a diameter/width of coil 270 so as to facilitate contact with hub 214. Band(s) 280 may be made from conductive material. Therefore, because band(s) 280 may contact hub 214, band(s) 280 may transmit electrical energy from hub 214 to coil 270. The energy may travel distally along coil 270 to electrode 220 to energize electrode 220.

Proximal channel 254 may be in fluid communication with distal channel 282, such that fluid from fluid port 112 may travel through proximal channel 254 and into lumen 234 of shaft 204. A proximal end of shaft 204 may be open to allow fluid to flow into a proximal end of lumen 234 of shaft 204 via proximal channel 254.

In use, medical device 100/200 may be used to deliver fluid to a treatment site. For example, fluid from a fluid source may be delivered through lumen 134/234 of shaft 104/204. Fluid may flow from lumen 134/234 of shaft 104/204 into a lumen (e.g., lumen 126) of electrode 120/220. In some aspects, the user may energize electrode 120/220 (using hub 114/214 to deliver energy through wire 132 or coil 270 to energize electrode 120) to cut one or more layers of tissue, and may then position distal tip 124/224 of electrode 120/220 between layers of tissue. The user may then deliver fluid through electrode 120/220, as discussed above, to deliver fluid between the layers of tissue. The fluid between the layers of tissue may help to separate the layers of tissue, elevate one or more upper layers of tissue, form a bleb, etc. The user may then energize electrode 120/220 again, for example, to further cut tissue or one or more other portions of the treatment site. During an entirety of the procedure, electrode 120/220 may have the same length. In other words, because electrode 120/220 is not capable of being actuated in a proximal or distal direction, electrode 120/220 may have an unchanging axial/longitudinal length throughout an entire procedure. Phrased differently, electrode 120/220 may be fixed relative to a distal end of shaft 104/204.

In one example, an electrosurgical generator coupled to the handle (or within the handle) may generate energy in various modes, for example, radio frequency energy in a cutting mode, a coagulation mode, etc., in order for the end effectors to deliver these different modes of energy to the tissue. In one aspect, the electrosurgical generator and/or the handle may include one or more knobs, dials, buttons, etc. in order to select the energy mode. Additionally, in one example, a fluid source (e.g., a saline source) coupled to the medical device may provide fluid (e.g., saline) to be delivered through the end effectors to the tissue and/or the treatment site. The fluid may be delivered at a constant rate, a pulsed rate, a user-controlled rate, etc. In these aspects, one or more of the energy delivery and/or the fluid delivery may be controlled by one or more actuators (e.g., triggers, buttons, touch screens, foot pedals, etc.).

The user may also deliver fluid distally out of the distal end of the end effectors (e.g., through the electrode), either simultaneously or sequentially with the energy delivered, which may help the user to more quickly and efficiently deliver the medical therapy, for example, cut, dissect, ablate, mark, coagulate, cauterize, or otherwise treat tissue. Moreover, the user may deliver fluid and energy without removing the medical device from the patient or subject, which may help to reduce the costs and duration of the procedure, also potentially reducing the risks to the subject.

While principles of the disclosure are described herein with reference to illustrative aspects for particular applications, it should be understood that the disclosure is not limited thereto. Those having ordinary skill in the art and access to the teachings provided herein will recognize additional modifications, applications, aspects, and substitution of equivalents all fall within the scope of the aspects described herein. Accordingly, the disclosure is not to be considered as limited by the foregoing description.

We claim:

1. A medical device for delivering energy and/or fluid, the medical device comprising:
a handle including:
a hub for electrically connecting the medical device to an energy source; and
a fluid port configured to receive fluid from a fluid source;
a shaft including a lumen for transmitting the fluid from the fluid source and a conductor for transmitting energy from the energy source; and
an electrode at a distal end of the shaft, wherein the electrode is electrically coupled to the conductor to deliver energy from the energy source to a tissue, wherein the electrode includes a lumen having a distal opening for delivering fluid from the fluid source to the tissue, and wherein the electrode is immovable relative to the distal end of the shaft.

2. The medical device of claim 1, wherein the shaft includes a sheath, wherein the sheath defines the lumen, and wherein the conductor is a wire that extends through the lumen.

3. The medical device of claim 1, wherein the conductor includes a coil.

4. The medical device of claim 3, wherein the coil extends from the handle to the distal end of the shaft.

5. The medical device of claim 3, wherein windings of the coil define the lumen of the shaft.

6. The medical device of claim 5, wherein the handle includes:
a proximal channel that is fluidly coupled to the fluid port; and
a distal channel, wherein a proximal portion of the coil is disposed within the distal channel.

7. The medical device of claim 6, wherein the distal channel is in fluid communication with the proximal channel, such that fluid is configured to flow from the fluid port, through the proximal channel, and into the lumen of the shaft.

8. The medical device of claim 3, wherein a distal end of the coil is electrically coupled to the electrode.

9. The medical device of claim 8, wherein an outer surface of a portion the coil includes an insulative coating.

10. The medical device of claim 9, wherein the insulative coating terminates proximally of the distal end of the coil.

11. The medical device of claim 3, wherein the coil is electrically coupled to the hub by a band.

12. The medical device of claim 11, wherein the band extends around at least a portion of a circumference of the coil.

13. The medical device of claim 1, further comprising a distal cap, wherein the distal cap includes an opening extending longitudinally through a portion of the distal cap, wherein a portion of the electrode extends through the opening of the distal cap.

14. The medical device of claim 13, wherein the electrode is immovable with respect to the distal cap.

15. The medical device of claim 1, wherein the electrode includes a widened distal end.

16. A medical device for delivering energy and/or fluid, the medical device comprising:
a shaft including a conductive coil, wherein the coil includes a plurality of windings wound around a central lumen; and
a distal end including an electrode, wherein the electrode is electrically coupled to the conductive coil, such that energy transmitted along the conductive coil energizes the electrode.

17. The medical device of claim 16, wherein the electrode includes a lumen having a distal opening, wherein the lumen of the electrode is in fluid communication with the central lumen of the shaft, such that a fluid flowing through the central lumen of the shaft flows into the lumen of the electrode and exits out of the distal opening of the electrode.

18. The medical device of claim 16, wherein a distal end of the coil contacts the electrode.

19. A medical device for delivering energy and/or fluid, the medical device comprising:
a shaft including a conductive coil, wherein the coil includes a plurality of windings wound around a central lumen; and
a handle including:
a hub for electrically connecting the medical device to an energy source; and
a fluid port configured to receive fluid from a fluid source;
wherein the hub is electrically coupled to a proximal portion of the conductive coil, and wherein the fluid port is in fluid communication with the central lumen of the shaft.

20. The medical device of claim 19, wherein the hub is electrically coupled to the coil via a conductive band that extends around the coil.
